(19) United States
(12) Patent Application Publication
(10) Pub. No.: US 2009/0258974 A1
(43) Pub. Date: Oct. 15, 2009

(54) OPTICALLY TRANSMISSIVE RESILIENT POLYMERS AND METHODS OF MANUFACTURE

(76) Inventors: EDWIN SLAGEL, Avondale, AZ (US); Michael Slagel, Peoria, AZ (US)

Correspondence Address: THE NOBLITT GROUP, PLLC, 4800 NORTH SCOTTSDALE ROAD, SUITE 6000, SCOTTSDALE, AZ, 85251, US

(21) Appl. No.: 12/366,813

(22) Filed: Feb. 6, 2009

Related U.S. Application Data
(60) Provisional application No. 61/026,587, filed on Feb. 6, 2008.

(51) Int. Cl. C08G 18/28; C08K 5/3475; C08K 5/17; C08K 5/13; C08G 18/00; C07C 265/00

(52) U.S. Cl. 524/91; 524/186; 524/352; 524/590; 528/64; 560/330

(57) ABSTRACT

In accordance with an exemplary embodiment of the present invention, an optically clear polyurethane/polyurea polymer may be prepared by reacting a cycloaliphatic diisocyanate with polyesters, polyethers, and polycarbonates which may comprise hydroxyl groups and/or primary amine groups. This reaction may be carried out using 0.5 to 0.9 equivalents of the hydroxyl/amine groups followed by the reaction of 0.1 to 0.5 equivalents of water. The glycol, amine, and water may then be reacted with a cycloaliphatic diisocyanate in a ratio of about 1.77 to about 3.5 NCO for each OH, HOH, NH₂. The resulting prepolymer may then be reacted with an aromatic diamine curing agent such as diethyltoluene diamine in an equivalent ratio of about 0.85 NH₂/1.0 NCO to about 1.1 NH₂/1.0 NCO, and in one embodiment, from about 0.90 NH₂/1.0 NCO to 0.98 NH₂/1.0 NCO. In one embodiment, 0.2 to 0.3 comprises the range of water equivalents for the water diisocyanate reaction.

### CROSS REFERENCES TO RELATED APPLICATIONS

This application claims the benefit of U.S. Provisional Patent Application No. 61/026,587, filed Feb. 6, 2008, and incorporates the disclosure of that application by reference.

### BACKGROUND OF INVENTION

Two increasingly important considerations in recent times are security and safety. For example, military vehicles generally require greater than average protection for its occupants. Such heightened protection gives rise to the need for various engineered transparent armor structures. For example, windshields and surrounding windows need to resist the incursion of small arms projectiles and shrapnel to protect its occupants, yet allow and maintain clear viewing of the outside environment by the occupants.

One class of plastics that proves both useful and reliable for constructing transparent armor structures is polycarbonate. Polycarbonate demonstrates superior characteristics for many applications to provide for overall protection because it maintains protective integrity within a wide range between its brittleness transition temperature and its heat distortion temperature. For example, applications such as transparent and/or translucent armor, vehicle glazings, architectural glazings, riot shields, aircraft canopies, face masks, visors, ophthalmic and sun lenses, protective eyewear and/or the like, may benefit from such polycarboiiate material.

Unfortunately, polycarbonate and other often used plastic materials are also relatively soft and unless hard-coated may easily abrade by its interaction with dirt and dust. As a result, these abrasions may adversely affect the optical properties of the polycarbonate and the transparency of the application can substantially degrade in a time span as short as one year. This substantial degradation of transparency generally necessitates replacement of the armor, and since transparent armor is expensive, frequent replacement creates a substantial financial burden on maintenance budgets. Furthermore, polycarbonate is often adversely affected by solvents and cleaning solutions when used to remove such dirt and dust, thereby exacerbating the transparency degradation.

### SUMMARY OF INVENTION

In accordance with exemplary embodiments, the present invention provides compositions and methods for high hardness polyurea and/or polyurethane polymers as an alternative to polycarbonate for transparent and/or transmissive applications. More particularly, the present invention provides for a water based urethane polymer wherein a water/diisocyanate reaction produces a chemical backbone comprising urea groups in a cycloaliphatic diisocyanate to improve heat resistance, chemical resistance, and impact resistance of the polymer while maintaining excellent transparent properties. Advantages of the present invention are set forth in the Detailed Description that follows, and may be apparent in view of the following Detailed Description or may be learned by practice of exemplary embodiments of the present invention.

### BRIEF DESCRIPTION OF THE DRAWINGS

Exemplary elements, operational features, applications and/or advantages of the present invention reside inter alia in the details of construction and operation as more fully hereafter depicted, described or otherwise identified—reference being made to the accompanying drawings, images, figures, etc. forming a part hereof (if any), wherein like numerals (if any) refer to like parts throughout. Other elements, operational features, applications and/or advantages will become apparent in view of certain exemplary embodiments recited in the disclosure herein.

FIGS. 1-3 are chemical diagrams of exemplary diisocyanates, in accordance with exemplary embodiments of the present invention;

FIGS. 4-6 are chemical diagrams of exemplary isomers of diamine curing agents, in accordance with exemplary embodiments of the present invention;

FIG. 7 is a chemical diagram of an exemplary antioxidant, in accordance with an exemplary embodiment of the present invention;

FIGS. 8-9 are chemical diagrams of exemplary U-V stabilizers, in accordance with exemplary embodiments of the present invention;

FIGS. 10-12 are chemical diagrams of exemplary hindered amine light stabilizers, in accordance with exemplary embodiments of the present invention; and

FIGS. 13-14 are flowcharts of an exemplary method for preparing a resulting polymer, in accordance with an exemplary embodiment of the present invention.

It will be appreciated that elements in the drawings, images, figures, etc. are illustrated for simplicity and clarity and have not necessarily been drawn to scale. For example, the dimensions of some of the elements in the figures may be exaggerated relative to other elements to help improve understanding of various embodiments of the present invention. Furthermore, the terms ‘first’, ‘second’, and the like herein, if any, are used inter alia for distinguishing betveen similar elements and not necessarily for describing a sequential or chronological order. Moreover, the terms ‘front’, ‘back’, ‘top’, ‘bottom’, ‘over’, ‘under’, and the like in the disclosure and/or in the provisional embodiments, if any, are generally employed for descriptive purposes and not necessarily for comprehensively describing exclusive relative position. It will be understood that any of the preceding terms so used may be interchanged under appropriate circumstances such that various embodiments of the invention described herein, for example, are capable of operation in other configurations and/or orientations than those explicitly illustrated or otherwise described.

### DETAILED DESCRIPTION OF EXEMPLARY EMBODIMENTS

The descriptions contained herein are of exemplary embodiments of the invention and the inventor's conception of the best mode, and are not intended to limit the scope, applicability, or configuration of the invention in any way. Rather, the following description is intended to provide convenient illustrations for implementing various embodiments of the invention. As will become apparent, changes may be made in the function and/or arrangement of any of the elements described in the disclosed exemplary embodiments without departing from the spirit and scope of the invention.

Exemplary embodiments of the present invention may be applied to any system for providing optically transmissive, high hardness polyurethane and/or polyurea polymers. Polyurethane and/or polyurea polymers in accordance with the present invention may comprise a substitute for polycarbonate in various transparent and/or transmissive applications that require superior resistance to: impacts, high heat distortion temperatures, and chemical degradation. The present invention may be generally described to comprise compositions and methods for providing water-based, high impact resistant, high heat resistant, and/or chemically resistant polyurea and/or polyurethane polymers. Such applications may comprise transparent armor, translucent armor, opaque armor, glazing applications, lenses, aircraft canopies, face masks, shields and/or the like. In one exemplary embodiment of the present invention, polyurethane and/or polyurea polymers may comprise highly resistant ballistic armor windshields that may optionally be tinted and/or color stabilized. In a further exemplary embodiment of the present invention, polyurethane and/or polyurea polymers may also be suitable for implementation in eyewear, such as ophthalmic lenses, sunglasses, and/or the like.

Polyurethane and/or polyurea polymers, in accordance with exemplary embodiments of the present invention, may comprise various beneficial characteristics over the prior art. For example, the addition of water to a reaction may be part of a process to create a prepolymer, where the prepolymer may be used to prepare a final resulting polymer, the unique prepolymer contributes, at least partially, to alter the failure characteristics of the resulting polymers during a heat distortion test. This alteration improves the overall thermal stability of the resulting polymer. In an exemplary embodiment of the present invention, a resulting polymer of a polyurethane and/or a polyurea may withstand heat distortion temperatures up to about 325° F. at 264 psi, more particularly, within a temperature range from about 315° F. to about 325° F. at 264 psi, and even more particularly, at least a temperature of 315° F. at 264 psi. The resulting polymer may further comprise a thickness/FSP (fragment simulating projectile) rating for a 0.25 inch thick sheet of a resulting polymer. The rating may comprise of a V-50 0.22 caliber FSP rating of at least 1150 feet per second. Furthermore, a resulting polymer may comprise a stress craze resistance of greater than about 7000 pounds per square inch when measured using isopropanol, and a resulting polyurethane and/or polyurea polymer may also comprise a luminous transmission of about at least 80%.

The present invention may be described in terms of prepolymers, aromatic diamine curing agents and/or the like. Prepolymers, according to various embodiments of the present invention, may comprise products of a reaction between at least one diisocyanate and at least one glycol reactant.

In accordance with an exemplary embodiment of the present invention, an optically clear polyurethane/polyurea resulting polymer of this invention may be prepared by reacting a cycloaliphatic diisocyanate with polyesters, polyethers, and/or polycarbonates that may comprise hydroxyl groups and/or primary amine groups. This reaction may be carried out using about 0.5 to about 0.9 equivalents of the hydroxyl/amine groups followed by the reaction of about 0.1 to about 0.5 equivalents of water. The glycol, amine, and water may then be reacted with a cycloaliphatic diisocyanate in a ratio of about 1.77 to about 3.5 NCO for each OH, HOH, NH₂. The resulting prepolymer may then be reacted with an aromatic diamine curing agent such as diethyltoluene diamine in an equivalent ratio of about 0.85 to about 1.1 NH₂/1.0 NCO, and in one embodiment, from about 0.90 to about 0.98 NH₂/1.0 NCO. In another exemplary embodiment, 0.2 to 0.3 may comprise the water equivalents range for the water-diisocyanate reaction. Dyes and/or pigments may also be added to achieve translucent, colored, and/or opaque polymers.

According to exemplary embodiments of the present invention, diisocyanates disclosed in this Detailed Description may comprise any organic compound that may comprise two isocyanate groups, for example, an isocyanate group may comprise (—N═C═O). In an exemplary embodiment of the present invention, diisocyanates may be synthesized from any desired molecular structures comprising amine functional groups, wherein such molecular structures may be reacted with, for example, phosgene, and wherein amine functional groups may be subsequently replaced with isocyanate groups. Suitable methods of synthesis of diisocyanates may comprise a Curtius rearrangement of acyl azides, a Lossen rearrangement of hydroxamic acids, and/or the like methods. Moreover, diisocyanates may comprise aliphatic and/or cycloaliphatic diisocyanates. In some exemplary embodiment of the present invention, diisocyanates may comprise dicyclohexylmethane diisocyanates.

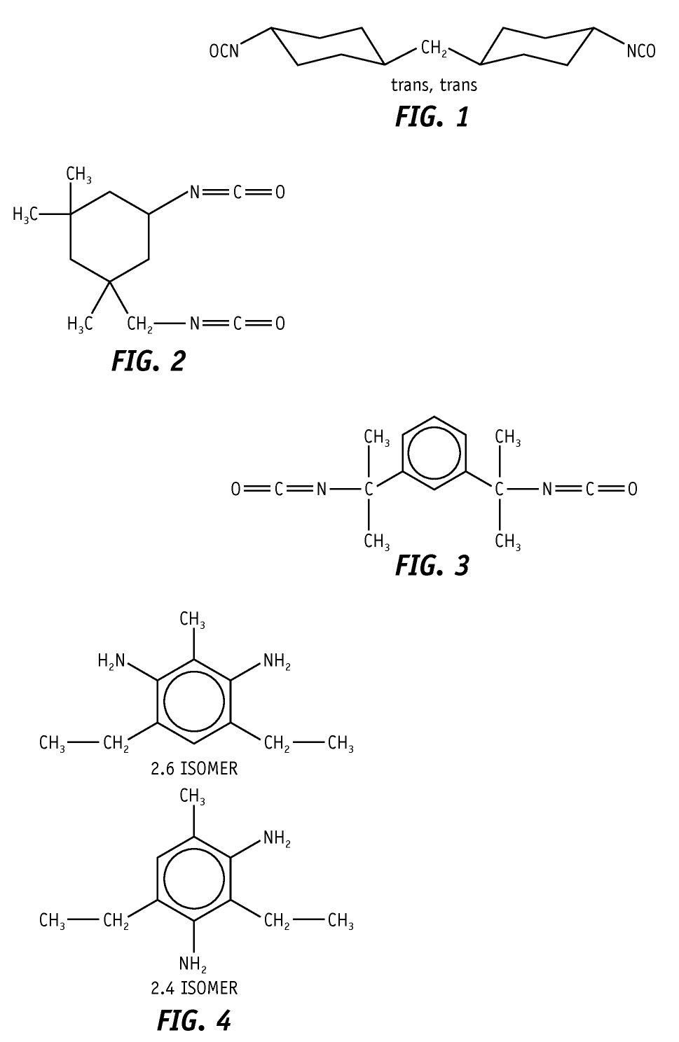

Referring now to FIG. 1, exemplary diisocyanates may comprise a trans,trans isomer of 4,4′-methylenebis(cyclohexyl isocyanate), which may also be referred to as a paraisocyanato cyclohexylmethane (“PICM”). The PICM may be synthesized by at least partially phosgenating 4,4′-methylenebis(cyclohexyl amine) molecules and/or the like. In one embodiment of the present invention, a presence of an at least partially high level of a trans,trans isomer of 4,4′-methylenebis(cyclohexyl isocyanate) may impart beneficial hardness properties as described. Moreover, in several exemplary embodiments, the beneficial properties described may increase with greater trans, trans concentration.

In accordance with another exemplary embodiment of the present invention, and referring now to FIG. 2, diisocyanates may comprise 3-isocyanato-methyl-3,5,5-trimethyl cyclohexyl-isocyanate (“IPDI”) available from Arco Chemical (Newtown Square, Pa., USA). In yet another embodiment of the present invention, referring now to FIG. 3, diisocyanates may also comprise a meta-tetramethylxylene diisocyanate (1,3-bis(1-isocyanato-1-methyl)-benzene), also known as TMXDI® (Meta) Aliphatic Isocyanate available from Cytec Industries, Inc. (West Paterson, N.J., USA).

Glycol reactants, according to exemplary embodiments of the present invention, may comprise compositions comprising at least one hydroxyl group and/or at least one primary amine group, and the glycol reactants may be substantially configured to react with the various diisocyanates and/or water. The glycol reactants may also comprise polyesters, polyethers, polycarbonates, and/or the like. In other exemplary embodiments of the present invention, the glycol reactants may comprise at least one of: esterification products of adipic acid comprising one or more diols comprising at least one of: 1,4-butanediol, 1,6-hexanediol, neopentyl glycol, and 1,10-decanediol; reaction products of ε-caprolactone with one or more diols comprising at least one of: 1,4-butane diol, 1,6-hexane diol, neopentyl clycol, and 1,10-decanediol; polytetramethylene glycol; aliphatic polycarbonate glycols and/or the like. According to exemplary embodiments, the glycol reactants may comprise an average molecular weight from about 90 to about 4000.

According to exemplary embodiments of the present invention, the glycol reactants may comprise glycols manufactured and/or sold by UBE America (New York, N.Y., USA), under trade names including UH-50, AME500, UH-100AMW1000, and UH200AMW2000 and/or the like. In another exemplary embodiment of the present invention, glycol reactants may comprise of a formula such as HO—[ROCOO]ₙ—R—OH, where R═(CH₂)₆.

The glycol reactants, in accordance with exemplary embodiments of the present invention, may also comprise at least one triol. A triol may comprise at least one of: a trimethylol ethane, a trimethylol propane, and/or the like, and the triols may be present in amounts sufficient to produce from about 0.1% to about 4% of the glycol reactants.

In accordance with exemplary embodiments of the present invention, a reaction between a diisocyanate and a glycol reactant may take place in the presence of water and/or a catalyst. Suitable catalysts in accordance with the present invention may comprise of an organo tin catalyst.

A reaction between diisocyanates and glycol reactants in accordance with the present invention may be carried out in any suitable manner. For example, in an exemplary embodiment of the present invention, a diisocyanate may be added to a reaction vessel, wherein the reaction vessel may be suitably coupled to an agitator. After addition of the diisocyanate to the reaction vessel, the glycol reactants (preheated to about 110° F. to about 130° F.) may be added to the reaction vessel. In another exemplary embodiment of the present invention, after addition of a diisocyanate and/or glycol reactants to a reaction vessel, an organo tin catalyst may be added. However, if the temperature is sufficient the catalyst may be eliminated. In exemplary embodiments of the present invention, about 20 ppm to about 40 ppm of the organo tin catalyst may be added to the diisocyanate and/or the glycol reactants in the reaction vessel. In other exemplary embodiments more or less of the catalyst may be added as needed to drive the reaction.

The catalysts in accordance with the present invention, such as organo tin catalysts, may be neutralized by addition of a phosphate comprising complexes, such as 1681 from Axel Research Laboratories, Inc. (Woodside, N.Y., USA). In an exemplary embodiment of the present invention, phosphate comprising complexes, such as 1681, may also function as an internal release agent to at least partially enhance subsequent de-molding of cast parts. Other neutralizers and/or releasing agents may also be used to achieve similar results.

In accordance with exemplary embodiments of the present invention, after addition of a catalyst to a vessel, reactants in the vessel may be allowed to exotherm to a range from about 230° F. to about 275° F. When the reactants in the vessel have cooled to a range from about 220° F. to about 255° F., a surfactant (or de-foamer) may be added to at least partially deplete any bubble formation which may occur in subsequent reactions in the vessel, such as during release of a CO₂ gas.

In accordance with exemplary embodiment of the present invention, water may be added after addition of the surfactant and/or after the reaction between the glycol reactants and the diisocyanate has cooled. Water may be added in a liquid and/or gas phase. In an alternate exemplary embodiment of the present invention, water may be added to a reaction vessel prior to the glycol reactants. In an exemplary embodiment of the present invention, cycloaliphatic diisocyanates comprising at least one NCO group bonded to a cycloaliphatic structure may be reacted with glycol reactants in amounts which provide a ratio of about 1.77 NCO to about 3.5 NCO for each hydroxyl group, amine group and/or water present in the glycol reactants. In yet a further embodiment, a ratio of about 0.2 NH₂ to about 0.3 NH₂ for 1.0 NCO may be suitable for a water diisocyanate reaction.

In accordance with exemplary embodiments of the present invention, a water-diisocyanate reaction may produce a backbone comprising urea groups (comprising a chemical formula of (NH₂)₂CO) in a cycloaliphatic diisocyanate. In an exemplary embodiment of the present invention, production of urea groups in a water/diisocyanate reaction may improve heat resistance, chemical resistance, and/or impact resistance of a prepolymer. In yet another exemplary embodiment of the present invention, production of urea groups in a water/diisocyanate reaction may at least substantially maintain transparency properties of a prepolymer. A prepolymer, in accordance with various embodiments of the present invention may be reacted with an aromatic diamine curing agent to produce a resulting high impact resistance and high heat distortion resistance polyurethane product.

In an exemplary embodiment of the present invention, an aromatic diamine curing agent may comprise diethyltoluene diamine in an equivalent ratio of about 0.85 NH₂ to about 1.1 NH₂ for 1.0 NCO. In another exemplary embodiment of the present invention, an aromatic diamine curing agent may comprise diethyltoluene diamine in an equivalent ratio of about 0.90 NH₂ to about 0.98 NH₂ for 1.0 NCO.

Diamine curing agents, in accordance with various exemplary embodiments of the present invention, may comprise any suitable aromatic diamine curing agent to prepare polyurethane and/or polyurea polymers. Referring now to FIG. 4, diamine curing agents may comprise diethyltoluene-diamines, such as 2,4-diamino-3,5-diethyl-toluene, and/or 2,6-diamino-3,5-diethyl-toluene. Diethyltoluene-diamines (“DETDA”) may appear under the trade name Ethacure 100 available from Albemarle Corporation (Baton Rouge, La., USA). General properties of DETDA include, for example, that it may be a liquid at room temperature and it may comprise a viscosity of about 156 centistokes (cS) at 25° C. Additionally, in an exemplary embodiment of the present invention, DETDA may comprise one or more isomers. In another exemplary embodiment of the present invention, DETDA may comprise 2,4-isomers in a range from about 75% to about 81% and 2,6-isomers in a range from about 18% to about 24%.

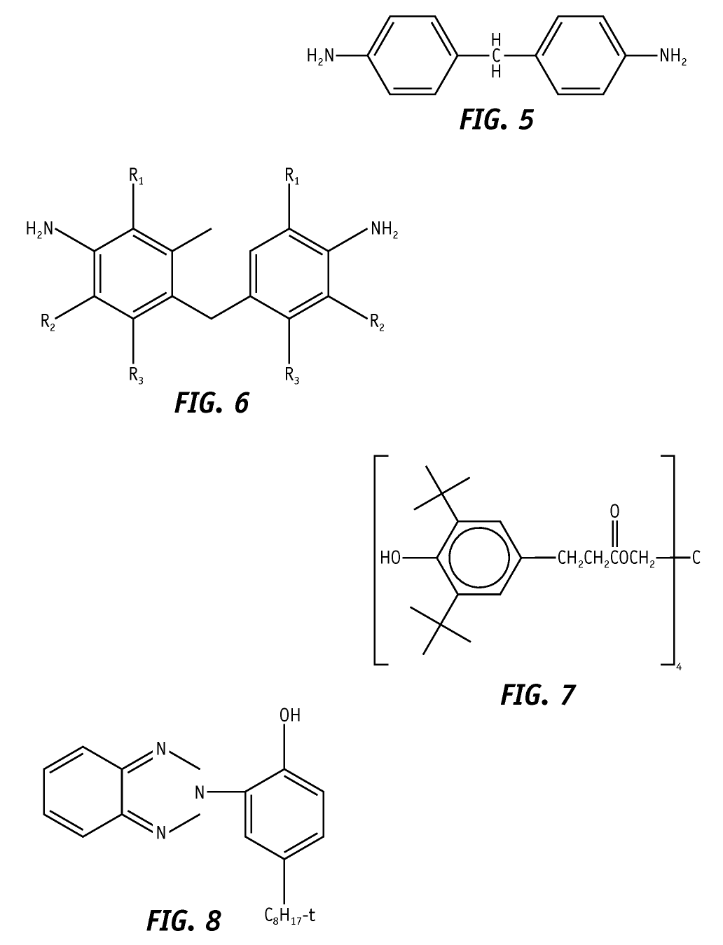

In an exemplary embodiment of the present invention, a diamine curing agent may comprise a color stabilized version of Ethacurc 100, which may be available under the name Ethacure 100LC available from Albemarle Corporation (Baton Rouge, La., USA). Referring now to FIGS. 5 and 6, diamine curing agents may comprise at least one of: 4,4′-methylene-bis(2,6-diisopropylaniline) (commercially known as LONZACURE M-DIPA and manufactured by Lonza Ltd. (Basel, Switzerland); 4,4′-methylene-bis(2,6-dimethylaniline) (commercially known as LONZACURE M-DMA and manufactured by Lonza Ltd. (Basel, Switzerland)); 4,4′-methylene-bis(2-ethyl-6-methylaniline) (commercially known as LONZACURE M-MEA and manufactured by Lonza Ltd. (Basel, Switzerland)); 4,4′-methylene-bis(2,6-diethylaniline) (commercially known as LONZACURE M-DEA and manufactured by Lonza Ltd. (Basel, Switzerland)); and/or 4,4′-methylene-bis(2-isopropyl-6-methylaniline) (commercially known as LONZACURE M-DIPA and manufactured by Lonza Ltd. (Basel, Switzerland)).

In accordance with exemplary embodiments of the present invention, various additional components may used to prepare polyurethane and/or polyurea polymers including but not limited to: antioxidants, ultraviolet stabilizers, color blockers, optical brighteners, mold release agents, and/or the like. Referring now to FIG. 7, in an exemplary embodiment of the present invention, suitable antioxidants may comprise multifunctional hindered phenol type antioxidants, such as Irganox 1010, available from Ciba Specialty Chemicals (Tarrytown, N.Y., USA), but other antioxidants may be used to achieve substantially similar results.

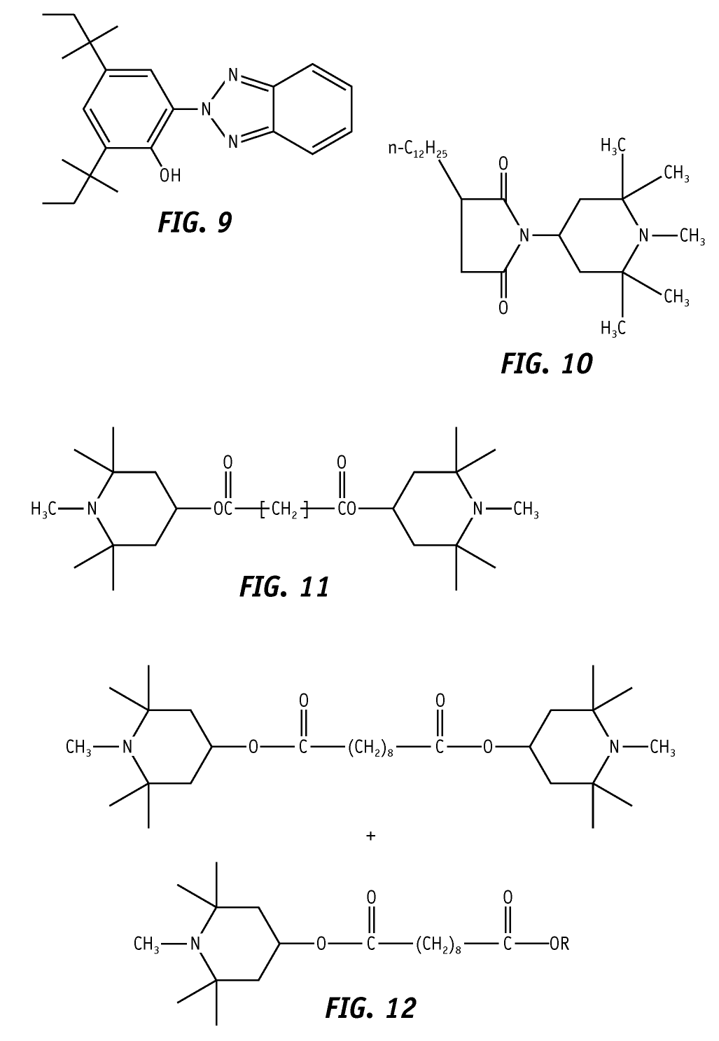

In an exemplary embodiment of the present invention, U-V stabilizers, comprising benzotriazoles may be used to prepare polyurethane and/or polyurea polymers. Referring now to FIGS. 8-9, exemplary U-V stabilizers in accordance with the present invention may comprise Cyasorb 5411, available from Ciba Specialty Chemicals (Tarrytown, N.Y., USA), and/or Tinuvin 328, available from Ciba Specialty Chemicals (Tarrytown, N.Y., USA). Other U-V stabilizers may also be used to achieve substantially similar results.

In another exemplary embodiment of the present invention, a hindered amine light stabilizer may be added to enhance, maintain, and/or provide UV protection. Referring now to FIGS. 10-12, suitable hindered amine light stabilizers in accordance with the present invention may comprise Cyasorb 3604, available from The Cary Company (Addison, Ill., USA); Tinuvin 765, available from Ciba Specialty Chemicals (Tarrytown, N.Y., USA); and/or a mixture of bis(1,2,2,6,6-pentamethyl-4-piperidinyl) sebacate and methyl(1,2,2,6,6-pentamethyl-4-piperidinyl) sebacate, also known as Lowilite® 92 available from Great Lakes Polymer Additives (West Lafayette, Ind., USA).

In accordance with exemplary embodiments of the present invention, additional components may be included in reactions that produce the prepolymers. An exemplary, non-inclusive list of additional components is identified in Table I below.

TABLE I

| | |
|---|---|
| CAPA 2047A | Solvay Interox (Houston, Texas USA) |
| CAPA 2107A | Solvay Interox (Houston, Texas USA) |
| CAPA 2407A | Solvay Interox (Houston, Texas USA) |
| CAPA 2077A | Solvay Interox (Houston, Texas USA) |
| UH-50 | UBE America (New York, New York, |
| | USA) |
| UH-100 | UBE America (New York, New York, |
| | USA) |
| UH-200 | UBE America (New York, New York, |
| | USA) |
| Ethacure 100LC | Albemarle Corporation (Baton Rouge, |
| | Louisiana, USA) |
| Desmodure W | Bayer Material Science, LLC |
| | (Pittsburgh, Pennsylvania, USA) |
| Tinuvin 328 | Ciba Specialty Chemicals (Tarrytown, |
| | New York, USA) |
| Tinuvin 765 | Ciba Specialty Chemicals (Tarrytown, |
| | New York, USA) |
| Irganox 1010 | Ciba Specialty Chemicals (Tarrytown, |
| | New York, USA) |
| Lowilite 92 | Great Lakes Polymer Additives (West |
| | Lafayette, Indiana, USA) |
| SF-8843 | GE |
| Exciton blue | Exciton (Dayton, Ohio, USA) |
| UL-28 | Compton |
| Acetocure-600 | Aceto Corp. (Japan) |

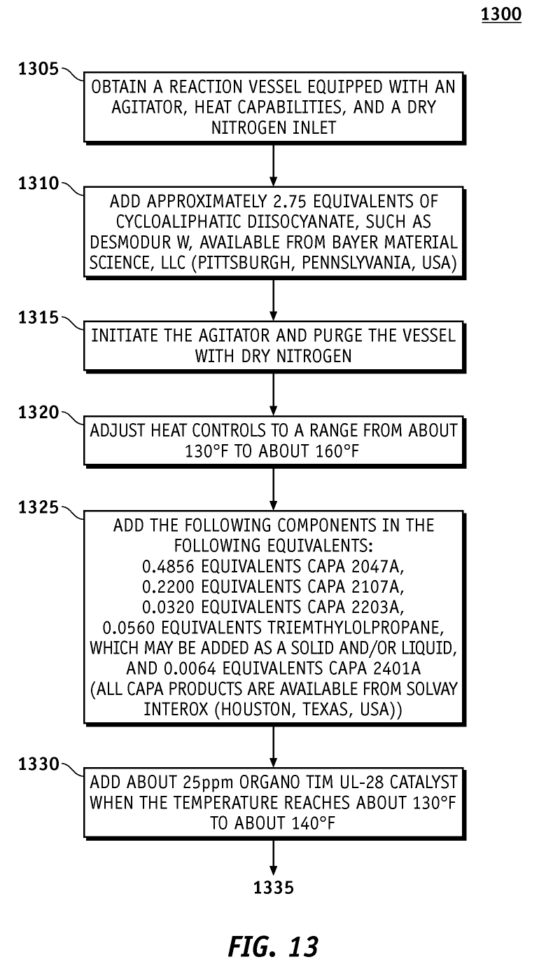

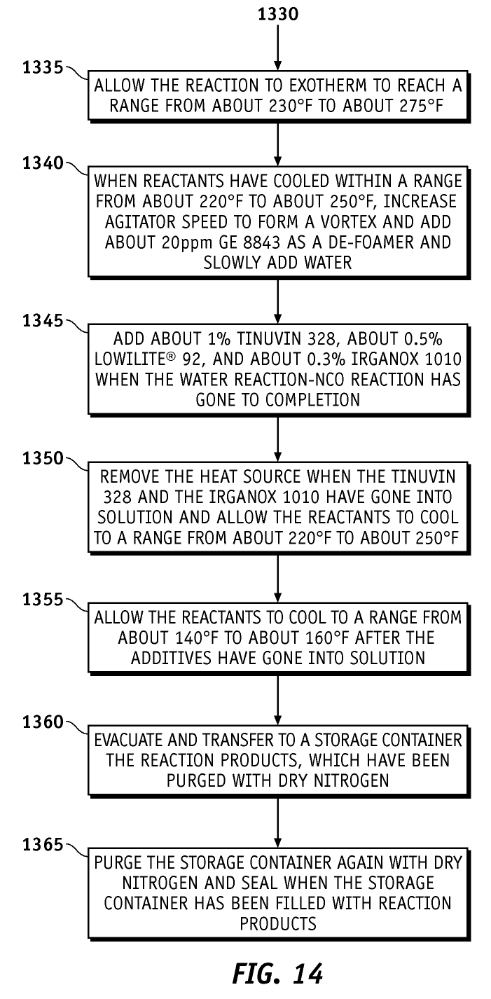

Prepolymers and diamine curing agents in accordance with the present invention may be implemented in various reaction protocols to produce a polyurea and/or a polyurethane polymer. For example, in one exemplary embodiment of the present invention and with reference to FIGS. 13-14, an exemplary method (1300) may comprise obtaining a reaction vessel equipped with an agitator, heat capabilities, and a dry nitrogen inlet (1305), and adding approximately 2.75 equivalents of cycloaliphatic diisocyanate, such as Desmodur W, available from Bayer Material Science, LLC (Pittsburgh, Pa., USA) (1310). The method may further comprise initiating the agitator and purging the vessel with dry nitrogen (1315); adjusting heat controls to a range from about 130° F. to about 160° F. (1320); and adding the following components in the following equivalents: 0.4856 equivalents CAPA 2047A, 0.2200 equivalents CAPA 2107A, 0.0320 equivalents CAPA 2203A, 0.0560 equivalents triemthylolpropane, which may be added as a solid and/or liquid, and 0.0064 equivalents CAPA 2401A (All CAPA products are available from Solvay Interox (Houston, Tex., USA)) (1325). The method may then comprise adding about 25 ppm organo tin UL-28 catalyst when the temperature reaches about 130° F. to about 140° F. (1330) and allowing the reaction to exotherm to reach a range from about 230° F. to about 275° F. (1335). When the reactants have cooled within a range from about 220° F. to about 250° F., increase agitator speed to form a vortex. Add about 20 ppm GE 8843 as a de-foamer and slowly add water (1340). When the water reaction-NCO reaction has gone to completion, add about 1% Tinuvin 328, about 0.5% Lowilite® 92, and about 0.3% Irganox 1010, (1345). The method may then further comprise removing the heat source when the Tinuvin 328 and the Irganox 1010 have gone into solution and allowing the reactants to cool to a range from about 220° F. to about 250° F. (1350). After the additives have gone into solution, allow the reactants to cool to a range from about 140° F. to about 160° F. (1355). The reaction products may then be evacuated and transferred to a storage container which has been purged with dry nitrogen (1360), and when the storage container has been filled with reaction products, purge the storage container again with dry nitrogen and seal (1365).

The described method is merely one exemplary method embodiment for preparing an optically clear polyurethane/polyurea polymer in accordance with the present invention. Some of the method steps may be altered, omitted or replaced by other method steps to achieve substantially similar results. For example, at sufficient temperatures, the addition of the catalyst may be omitted. In other examples, additives different than those specifically mentioned may be used to achieve substantially similar results.

Exemplary properties of a resulting reaction product in accordance with the present invention may be summarized by the following Table II:

TABLE II

| | |
|---|---|
| Shore D hardness | 83-84 |
| Heat distortion 264 fiberstress, F. | 320 |
| Tensile strength (psi) | 7500 |
| Elongation (%) | 140 |
| Modulus (kpsi) | 190 |
| Color | Very low color |

With regard to the Table II above, as well as with respect to the present invention, heat distortion may be defined as deflection of about 0.010 inches with a temperature increase of about 2° C. per minute. In accordance with the present invention, it has been shown that by reacting water with the diisocyanate, as described, an improvement in the heat distortion failure mode is achieved. The heat distortion of similar systems without the addition of water shows a rapid fall off in deflection, about 6-7 mils at low temperatures (about 140° F. to about 170° F.), then slowly deflects to about 10 mils, which is failure. The addition of water to the backbone shows a completely different failure mode, e.g., no deflection takes place until about 260° F. to about 275° F. before progressing to failure. This increases the heat resistance for ballistic impact.

Particular implementations shown and described herein are illustrative of the invention and its best mode and are not intended to otherwise limit the scope of the present invention in any way. Indeed, for the sake or brevity, prepolymers, diamine curing agents, polyurethanes, polyureas and/or the like may not be described in complete detail herein.

In the foregoing specification, the invention has been described with reference to specific exemplary embodiments; however, it will be appreciated that various modifications and changes may be made without departing from the scope of the present invention as set forth in the exemplary provisional embodiments. The specification and figures are to be regarded in an illustrative manner, rather than a restrictive one and all such modifications are intended to be included within the scope of the present invention. Accordingly, the scope of the invention should be determined by the provisional embodiments and their legal equivalents. For example, the steps recited in any method or process embodiments may be executed in any order and are not limited to the specific order presented in the provisional embodiments. Additionally, the components and/or elements recited in any apparatus embodiment may be assembled or othervise operationally configured in a variety of permutations to produce substantially the same result as the present invention and are accordingly not limited to the specific configuration recited in the provisional embodiments.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components of any or all the provisional embodiments.

As used herein, the terms “comprising”, “having”, “including”, or any contextual variant thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted by those skilled in the art to specific environments, manufacturing specifications, design parameters or other operating requirements without departing from the general principles of the same.

We claim:

1. A prepolymer composition for an impact resistant polyurethane material comprising the reaction product of:
at least one of a hydroxyl and an amine group from at least one of a polyester, a polyether, and a polycarbonate, wherein about 0.5 to about 0.9 weight equivalents of the at least one of the hydroxyl and the amine group; and
about 0.1 to about 0.5 weight equivalents of a water are reacted with a cycloaliphatic diisocyanate in a ratio of about 1.77 to about 3.5 for each OH, HOH, and NH₂ from the at least one of the hydroxyl and the amine group.

2. The composition of claim 1, further comprising about 20 ppm to about 40 ppm of an organo tin catalyst.

3. The composition of claim 2, further comprising about 20 ppm to about 40 ppm of a surfactant to deplete a bubble formulation during the water and the diisocyanate reaction.

4. The composition of claim 1, wherein the weight equivalents of the water comprises from about 0.2 to about 0.3.

5. The composition of claim 1, wherein a weight average molecular weight of the at least one of the hydroxyl and the amine group comprises from about 90 to about 4000.

6. The composition of claim 1, wherein the diisocyanate is selected from the group comprising a 4,4′-methylenebis(cyclohexyl isocyanate), a 3-isocyanato-methyl-3,5,5-trimethyl cyclohexyl-isocyanate, and a meta-tetramethylxylene diisocyanate (1,3-bis(1-isocyanato-1-methyl)-benzene).

7. The composition of claim 1, wherein the at least one of the hydroxyl and the amine group comprises at least one selected from the group comprising: (a) esterification products of adipic acid with one or more diols selected from the group comprising: 1,4-butanediol, 1,6-hexanediol, neopentyl glycol, and 1,10-decanediol; (b) reaction products of ε-caprolactone with one or more diols comprising selected from the group comprising: 1,4-butane diol, 1,6-hexane diol, neopentyl clycol, and 1,10-decanediol; (c) polytetramethylene glycol; (d) aliphatic polycarbonate glycols; and (e) mixtures of such at least one of the hydroxyl and the amine group.

8. A transparent, impact resistant polyurethane material comprising the reaction product of:
(a) a prepolymer comprising:
at least one of a hydroxyl and an amine group from at least one of a polyester, a polyether, and a polycarbonate, wherein about 0.5 to about 0.9 weight equivalents of the at least one of the hydroxyl and the amine group; and
about 0.1 to about 0.5 weight equivalents of a water are reacted with a cycloaliphatic diisocyanate in a ratio of about 1.77 to about 3.5 for each OH, HOH, and NH₂ from the at least one of the hydroxyl and the amine group; and
(b) at least one diamine curing agent selected from the group comprising a 2,4-diamino-3,5-diethyl-toluene, and a 2,6-diamino-3,5-diethyl-toluene in an equivalent ratio from about 0.85 NH₂/1.0 NCO to about 1.1 NH₂/1.0 NCO.

9. The polyurethane material of claim 8, further comprising about 20 ppm to about 40 ppm of an organo tin catalyst.

10. The polyurethane material of claim 8, further comprising about 20 to about 40 ppm of a surfactant to deplete a bubble formulation during the water and the diisocyanate reaction.

11. The polyurethane material of claim 8, wherein the weight equivalents of the water comprises from about 0.2 to about 0.3.

12. The polyurethane material of claim 8, wherein a weight average molecular weight of the at least one of the hydroxyl and the amine group comprises from about 90 to about 4000.

13. The polyurethane material of claim 8, wherein the diisocyanate is selected from the group comprising a 4,4′-methylenebis(cyclohexyl isocyanate), a 3-isocyanato-methyl-3,5,5-trimethyl cyclohexyl-isocyanate, and a meta-tetramethylxylene diisocyanate (1,3-bis(1-isocyanato-1-methyl)-benzene).

14. The polyurethane material of claim 8, wherein the at least one of the hydroxyl and the amine group comprises at least one selected from the group comprising: (a) esterification products of adipic acid with one or more diols selected from the group comprising: 1,4-butanediol, 1,6-hexanediol, neopentyl glycol, and 1,10-decanediol; (b) reaction products of ε-caprolactone with one or more diols comprising selected from the group comprising: 1,4-butane diol, 1,6-hexane diol, neopentyl clycol, and 1,10-decanediol; (c) polytetramethylene glycol; (d) aliphatic polycarbonate glycols; and (e) mixtures of such at least one of the hydroxyl and the amine group.

15. The polyurethane material of claim 8, wherein the polyurethane material comprises a heat distortion temperature in the range of 280° F. to 325° F. at 264 psi.

16. The polyurethane material of claim 8, wherein the polyurethane material comprises a heat distortion temperature of at least 280° F. at 264 psi.

17. The polyurethane material of claim 8, wherein the polyurethane material comprises a heat distortion temperature of at least 315° F. at 264 psi.

18. The polyurethane material of claim 8, wherein the polyurethane material is optically clear comprising a luminous transmittance of at least about 80%.

19. The polyurethane material of claim 8, wherein a 0.25-inch thick sheet of the polyurethane material comprises a V-50 0.22 caliber FSP rating of at least 1150 feet per second.

20. The polyurethane material of claim 8, wherein the diamine curing agent is reacted with the prepolymer in an equivalent ratio of about 0.90 to 1.0 NH₂/1.0 NCO.

21. The polyurethane material of claim 8, wherein the prepolymer further comprises a UV-stabilizer.

22. The polyurethane material of claim 21, wherein the UV-stabilizer is selected from the group comprising benzotriazoles, hindered amine light stabilizers, and mixtures thereof.

23. The polyurethane material of claim 8, wherein the prepolymer further comprises an anti-oxidant.

24. The polyurethane material of claim 23, wherein the anti-oxidant comprises a multifunctional hindered phenol.

25. The polyurethane material of claim 8, wherein the polyurethane material has a stress craze resistance of >7000 pounds per square inch when measured using isopropanol.

26. The polyurethane material of claim 8, wherein said diamine curing agent is color-stabilized.

27. A method for preparing a transparent, impact resistant polyurethane material comprising:
reacting a cycloaliphatic diisocyanate with about 0.5 to about 0.9 equivalents of hydroxyl/amine groups from at least one of a polyester, a polyether, and a polycarbonate and about 0.1 to about 0.5 equivalents of water;
reacting the cycloaliphatic diisocyanate in a ratio of about 1.77 to about 3.5 NCO for each OH, HOH, NH₂ to form a resulting prepolymer; and
reacting the resulting prepolymer with an aromatic diamine curing agent in an equivalent ratio from about 0.85 NH₂/1.0 NCO to about 1.1 NH₂/1.0 NCO.

28. The method of claim 27, wherein the aromatic diamine curing agent comprises diethyltoluene diamine.
